(19) United States
(12) Patent Application Publication
(10) Pub. No.: US 2025/0130215 A1
(43) Pub. Date: Apr. 24, 2025

(54) Climate Smart Agriculture System and Method

(71) Applicant: Kent Cavender-Bares, Minneapolis, MN (US)

(72) Inventor: Kent Cavender-Bares, Minneapolis, MN (US)

(21) Appl. No.: 18/926,058

(22) Filed: Oct. 24, 2024

Related U.S. Application Data
(60) Provisional application No. 63/592,801, filed on Oct. 24, 2023.

(51) Int. Cl. G01N 33/00; G01N 33/24

(52) U.S. Cl. CPC G01N 33/0036; G01N 33/0016; G01N 33/24

(57) ABSTRACT

A system and method for measuring gas emissions from a particular sample of soil. A climate smart system can be deployed in an agricultural field to monitor changes to greenhouse gas emissions from the soil. The climate smart system can operate independently of human direction and automatically collect samples from a series of locations within the agricultural field.

### CROSS-REFERENCE TO RELATED APPLICATION

The present disclosure claims the benefit of U.S. Provisional Application No. 63/592,801, filed Oct. 24, 2023, which is hereby fully incorporated by reference in its entirety.

### TECHNICAL FIELD

The present disclosure relates generally to a system and method for measuring gas emissions, and more particularly to a system and method for measuring greenhouse gas emissions in agricultural fields.

### BACKGROUND

Climate change presents many risks to humans, ecosystems, and agricultural productivity. As society works to meet goals to limit temperature increases due to climate change, measurement, reporting and verification of greenhouse gas mitigation efforts is essential. Emissions of greenhouse gases, such as nitrous oxide from agricultural fields, leads to greenhouse warming of the planet. Farming practices that accumulate atmospheric carbon dioxide as organic matter in soils offer a solution for reducing atmospheric greenhouse gases and the impacts of climate change. With government and private sector programs designed to rewards producers for “climate-smart” farming practices, improved characterization of greenhouse gas fluxes is essential.

Physical monitoring of greenhouse gas fluxes associated with crop fields has been largely constrained to smaller-scale research fields. Small chambers have been used extensively to measure greenhouse gas fluxes at the soil surface on crop fields. A chamber is typically attached to a base that has been installed at a location for the duration of the sampling period. To improve spatial representation, a number of bases can be installed across a field, although substantial labor is required to move chambers from base to base. Measurements of greenhouse gases can be made at the chamber using a portable, flow-through gas analyzer or gas samples can be extracted and analyzed later using a laboratory gas chromograph. This method is insufficient because it is extremely challenging to get both high spatial and temporal resolution data for an entire field. Researchers also have used eddy-flux towers to characterize greenhouse gas fluxes continuously at the scale of entire fields. These eddy-flux towers are cost prohibitive and require a dedicated installation, which can interfere with farming practices.

There is a need for a system and method capable of quantifying greenhouse gas fluxes on crop fields with both high temporal and spatial resolution. Achieving quantification through traditional methods is difficult by the inherent variability of in soils, rainfall, topography, cropping, and nutrient management practices. The current methods are cost prohibitive and are limited spatially and temporally. What is required is a low-cost solution that enables monitoring greenhouse gas fluxes with greater spatial and temporal resolution through frequent, chamber-based flux measurements at multiple points across a given field.

### SUMMARY

One embodiment of the present disclosure provides a climate smart agricultural system deployable into agricultural fields to collect samples of gases released by the soil of such agricultural fields. The system generally comprises an auto-sampling assembly and a housing. In one embodiment, the system is deployed onto a base, where the base is installed directly into an area of soil in the agricultural field in a fixed position. In another embodiment, the system is deployed directly onto an area of soil.

The summary above is not intended to describe each illustrated embodiment or every implementation of the present disclosure. The figures and the detailed description that follow more particularly exemplify these embodiments.

### BRIEF DESCRIPTION OF THE DRAWINGS

The disclosure can be more completely understood in consideration of the following detailed description of various embodiment of the disclosure, in connection with the accompanying drawings, in which:

FIG. 1 is a perspective view depicting an auto-sampling assembly, in accordance with a first embodiment of the disclosure;

FIG. 2A is a top-down perspective view of a syringe pump in a collection position, in accordance with an embodiment of the disclosure;

FIG. 2B is a side perspective view of a syringe pump in a injection position, in accordance with an embodiment of the disclosure.

While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION OF THE DRAWINGS

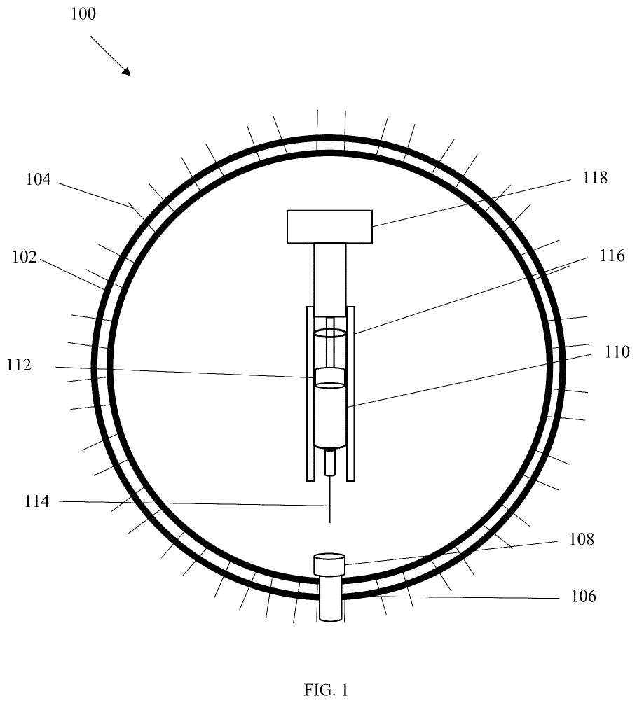

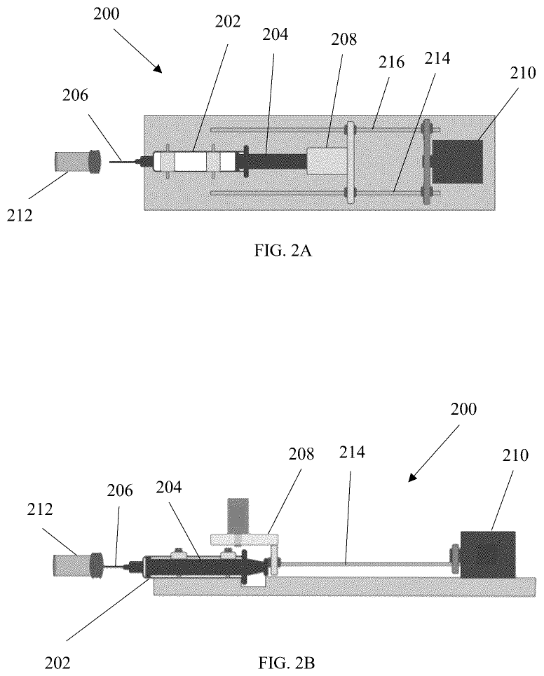

Referring to FIGS. 1, 2A, and 2B, a climate smart system and a climate smart method are disclosed in accordance with an embodiment of the disclosure. In embodiments, the climate smart system comprises an auto-sampling assembly and a housing for the auto-sampling assembly, wherein the housing is configured to interact with a base or an area of soil.

FIG. 1 depicts an embodiment of an auto-sampling assembly 100. The auto-sampling assembly 100 comprises a sample tray 102, a sample vial 106, and a syringe 110. The sample tray 102 is comprises a series of channels 104 configured to secure the sample vial 106 to the sample tray 102 during operation of the climate smart system 100. The sample vial 106 is configured to receive a sample cap 108.

The syringe 110 comprises a plunger 112 and a needle 114. The syringe 110 can be housed in a syringe pump 116, where the syringe pump 116 can move the syringe 110 laterally. The plunger actuator 118 actuates the plunger 112 of the syringe 110 to draw a sample into or eject the sample out of the syringe 110 via the needle 114.

To retrieve the sample, the syringe 110 is in a “filling” position, where no sample vial 106 is aligned with the needle 114. At a required time, the plunger actuator 118 withdraws the plunger 112, such a sample of air is drawn into the syringe 110. Once the sample has been collected, the sample tray 102 rotates to a “collection” position, such that the sample vial 106 is aligned with the needle 114. Once in the “collection” position, the syringe pump 116 actuated the syringe 110 such that the needle 114 is inserted through the sample cap 108 and into the sample vial 106. The plunger actuator 118 drives the plunger 112 into the syringe 110, ejecting the collected sample into the sample vial 106. After injection, the needle 114 retracts from the sample vial 106 and through the sample cap 108. The sample tray 102 is rotated to return to a “filling” position and the syringe 110 is fully closed to prepare for another sample.

In embodiments, the sample cap 108 comprises an elastomeric material. Suitable elastomeric materials are those that are self-sealing, such that when the needle 114 is withdrawn after the injection of the sample, the sample cap 108 is gastight and the collected sample does not escape. Such materials may include various elastomers. The sample vial can be evacuated prior to collection and can have a slight positive pressure of an inert gas added.

The climate smart system can be configured to collect a larger volume of sample into the syringe 110 than the volume of the sample vial 106. Injecting a larger volume of the sample than the sample vial 106 can hold ensures that ambient air does not leak into the sample vial 106 after collection and before sample analysis.

In embodiments, the climate smart system further comprises a computer, a motor, a belt, and a battery. The belt is affixed to the sample tray 102. The motor drives the belt such that the sample tray 102 is rotated to the required positions, including the “collection” position and the “filling” position described supra. The computer can be equipped with software configured to control the auto-sampling assembly of FIG. 1. The computer can control the syringe pump 116 and the plunger actuator 118 to initiate both collection and filling positions of the syringe 110 and the needle 114. The computer may further be functionally connected to a series of limit switches, wherein the limit switches indicate when the syringe 110 is fully closed (i.e. the plunger 112 is fully compressed into the syringe 110) and the needle 114 is fully extended (i.e. inserted into the sample vial 106). The computer can be configured to collect data to provide an electronic record of when the sample was collected and the configuration of the climate smart system. The computer can also be configured to remotely connect with broadband hotspots and virtual private networks such that the computer, including functionality and collected data, can be accessed remotely.

In certain embodiments, the climate support system further comprises a solar panel system functionally connected to the battery of the climate support system, such that the solar panel system recharges the battery.

FIG. 2A and FIG. 2B depict an embodiment of a syringe pump 200 suitable for the climate smart system disclosed herein. The syringe pump 200 comprises a syringe 202, a stepper motor 210, a short-distance linear actuator 208, a first fixed screw shaft 214, and a second fixed screw shaft 216. The syringe comprises a plunger 204 and a needle 206. The plunger 204 is connected to the linear actuator 208, such that the linear actuator can drive the plunger into the syringe 202 or withdraw the plunger 204 from the syringe 202.

FIG. 2A depicts the syringe pump 200 in the “collection” position, in which a sample has been collected into the syringe 202. FIG. 2B depicts the syringe pump in the “filling” position, in which the needle 206 is inserted into a sample vial 212 and the plunger 204 is fully depressed, thereby injecting the sample into the sample vial 212. The stepper motor 210 engages with the linear actuator 208 via the first fixed screw shaft 214 and the second fixed screw shaft 216, such that the linear actuator can depress and withdraw the plunger as described supra. In some embodiments, the computer of the climate smart system can monitor the current of the stepper motor 210 to determine if the syringe 202 is fully closed and the needle 206 is fully extended.

In some embodiments, the climate smart system comprises a base installed over an area of soil. The base comprises a chamber configured to mate with the housing of the climate smart system. The housing can comprise a gasket, such that when the housing mates with the base, a gastight or near gastight connections exists, in order to minimize fluctuations in gases during sample collection. The housing of the climate smart system can also be configured to mate directly with an area of soil. In such an embodiment, the housing comprises a gasket and an additional sealing component. The additional sealing component can be a weighted, flexible layer, configured to conform with a surface of the area of soil.

In some embodiments, the climate smart system further comprises a movement mechanism, configured to move the climate smart system from one base or area of soil to another discrete base or area of soil. It is contemplated that the movement mechanism is configured to move the climate smart system laterally and/or vertically. The movement mechanism can comprise a rolling housing support and a series of wheels. In such embodiments, the movement mechanism moves the housing, containing the auto-sampling assembly, over a base or an area of soil.

In embodiments where the climate smart system further comprises a base installed over an area of soil, it can be necessary to estimate the volume of the base above the area of soil because the surface of the area of soil can be uneven. In such embodiments, a board comprising a grid of holes can be placed over the base, wherein measurements to the surface of the soil from each of the holes can be made. An average of the measurements of the depth to the surface of the soil from each hole can be used to estimate the volume air over the area of soil. It is contemplated that this method can be automated by use of a camera, which can be affixed within the housing of the climate smart system.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numbers additional embodiments. Moreover, while various materials, dimensions, shapes, configurations, and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrate in an y individual embodiment described above. The embodiments described herein are not meant to be an exhaustive presentation of the ways in which the various features of the subject matter hereof may be combined. Accordingly, the embodiments are not mutually exclusive combinations of features; rather the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claims with the subject matter of each dependent or a combination of one or more features with other dependent or independent claims. Such combinations are proposed herein unless it is stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims, it is expressly intended that the provisions of 35 U.S.C. § 112(f) are not to be invoked unless the specific terms “means for” or “step for” are recited in a claim.

1. A greenhouse gas emissions monitoring system, the system comprising:
a housing defining an internal cavity;
a sampling system, the sampling system comprising a sample tray, at least one sample container, and a syringe, wherein the syringe comprises a needle and a plunger, and wherein the sampling system is housed within the internal cavity.

2. The monitoring system of claim 1, the system further comprising a movement mechanism configured to move the monitoring system laterally and vertically.

3. The monitoring system of claim 1, the sampling system further comprising:
a motor configured to directly or indirectly drive the sample tray;
a first actuator configured to actuate the plunger of the syringe, wherein actuation draws an air sample into the syringe and expels the air sample into the at least one sample container;
a second actuator configured to actuate the syringe, wherein the such that the needle is drawn and out of the sample container without releasing air pressure in the sample vial;
a computer, wherein the computer is configured to send and receive signals, store electronic data, and access a network.

4. The monitoring system of claim 1, wherein the monitoring system further comprises a base, wherein the base is installed directly into an area of soil and configured to removably couple with the housing of the monitoring system.

5. The monitoring system of claim 1, wherein the housing further comprises a gasket, wherein the gasket is configured to form a functionally airtight seal when the monitoring system is deployed onto the base.

6. The monitoring system of claim 1, wherein the movement mechanism comprises a housing support frame and a series of wheels.

7. The monitoring system of claim 1, wherein the sample tray has a zero position and a series of injection positions and wherein the injection positions are configured to align the at least one sample container with the needle of the syringe.

8. The monitoring system of claim 1, wherein the at least one sample container comprises a self-sealing lid such that the sample container is airtight after a sample has been injected into the sample container.

9. The monitoring system of claim 1, the monitoring system further comprising a battery functionally connected to the electronic components of the monitoring system.

10. The monitoring system of claim 1, the monitoring system further comprising a camera system, the camera system configured to estimate a volume of air above the area of soil confined by the base of the monitoring system.

11. The monitoring system of claim 1, wherein the sample tray is removably connected to the sampling system such that the sample tray can be replaced by a second sample tray with a plurality of empty sample containers.

12. A method of monitoring greenhouse gas emissions, the method comprising:
moving a greenhouse gas monitoring system into a position over an area of soil, wherein the monitoring system comprises a housing, a syringe, and a sample vial and wherein the syringe comprises a needle and a plunger;
actuating the plunger of the syringe such that the syringe draws in a sample,
inserting the needle of the syringe into a sample vial;
injecting the sample into the sample vial;
removing the needle from the sample vial.

13. The method of monitoring greenhouse gas emissions of claim 12, the method further comprising:
housing the sample vial in a sample tray; and
moving the sample tray of the greenhouse gas monitoring system from a zero position to an injection position, wherein the injection position aligns the sample vial with the needle of the syringe; and
replacing the sample tray with a second sample tray, the second sample tray comprising a plurality of unfilled sample vials.

14. The method of monitoring greenhouse gas emission of claim 12, wherein the monitoring system is moved into the position and deployed onto the base via a movement mechanism comprising a support frame and a set of wheels, the movement mechanism configured to move the monitoring system laterally and vertically.

15. The method of monitoring greenhouse gas emissions of claim 12, the method further comprising deploying the monitoring system onto a base, wherein the base confines the area of soil and wherein a gastight seal is formed between the housing and the base.

16. The method of monitoring greenhouse gas emissions of claim 12, wherein the greenhouse gas monitoring system is deployed in an agricultural field.

17. The method of monitoring greenhouse gas emissions of claim 12, the method further comprising measuring a volume of air above the area of soil confined by the base of the greenhouse gas monitoring system.

18. The method of monitoring greenhouse gas emissions, the method further comprising removing the sample vial from the greenhouse gas monitoring system and testing the collected sample for greenhouse gas emissions.

19. The method of monitoring greenhouse gas emissions, the method further comprising evacuating the sample vials prior to deploying the monitoring system and adding an inert gas such that the sample vial has a slight positive pressure.

20. The method of monitoring greenhouse gas emissions, the method further comprising collecting data about an instance of sample collection, including a time of a sample collection and a configuration of the monitoring system.
